(19) United States
(12) Patent Application Publication
(10) Pub. No.: US 2013/0035718 A1
(43) Pub. Date: Feb. 7, 2013

(54) FLEXIBLE FORCEPS WITH IMPROVED TORSIONAL RIGIDITY

(71) Applicant: James K. Brannon, Leawood, KS (US)

(72) Inventor: James K. Brannon, Leawood, KS (US)

(21) Appl. No.: 13/197,476

(22) Filed: Aug. 3, 2011

(51) Int. Cl. A61B 17/28

(52) U.S. Cl. 606/205

(57) ABSTRACT

An improved medical transmission apparatus for transmitting a flexible forceps through one of a plurality of lumens associated with an elongated composite scope assembly towards a surgical site presenting an increased torsional rigidity region for increased deflectional resistance of the forceps and being defined by a junction between a sheath associated with the forceps and a lumen extending cylindrically along the elongated composite scope assembly; a portal entry associated with the elongated composite scope assembly having a diameter sized for receiving and transmitting a distal tip section associated with said forceps therethrough and a cylindrical clearance positioned between an outer sidewall associated with said sheath and an inner surface associated with said lumen, the cylindrical clearance directing said forceps towards the surgical site.

### BACKGROUND OF THE INVENTION

The present invention is broadly directed to improvements in flexible forceps for use in endoscopic surgery and, more particularly, to such improvements which enable a surgeon to more positively position and orient forceps jaws at a surgical site.

Surgery is a learned art requiring many hours of advanced training and skills development that extends far beyond a thorough understanding of the medical principals involved, e.g., anatomy, physiology, principals of wound healing, and the like. The surgeon must also develop hand to eye coordination and acquire skills in the art of tissue manipulation utilizing a variety of highly specialized surgical instruments. The surgical instrument becomes an extension of the surgeon's hand. The surgeon must develop an ability to feel and respond, often delicately yet firmly and positively through his surgical instruments. Accordingly, there exists a need for surgical instruments which are sensitive, responsive, and ergonomically designed to augment the natural motions of the surgeon's hand.

Modern surgery tends toward minimally invasive techniques whenever possible. Although often more complicated in some ways for the surgeon, minimally invasive techniques result in less trauma to the patient and less scarring because of much smaller incisions thereby promoting faster healing and reducing possibilities for infections. In general, minimally invasive surgeries involve making one or more small incisions at appropriate locations and inserting tubular devices through the incisions to the surgical site. The tubular devices may be referred to as endoscopes, arthroscopes, laparoscopes, and the like and often have optical fiber based optical viewing apparatus and light sources, surgical instruments, lumens for inserting instruments or exchanging fluids with the surgical site, or combinations thereof extending therethrough. In some circumstances it is more appropriate to separate the light source and viewing scope from specifically surgical instruments, thus requiring two incisions and endoscopes. This technique is sometimes referred to as triangulation. In other instances, external types of imaging techniques are used for locating endoscopic instruments, such as fluoroscopes, computed tomography, magnetic resonance imaging, or the like.

Endoscopic instruments are configured in a number of different ways depending on their intended purpose. There are rigid endoscopes and flexible endoscopes. Some are simply tubes or portal instruments which provide access to a surgical site for instruments which are passed through the scopes or for the exchange of fluids to and from the surgical site. Viewing scopes, including light sources, may be used for viewing a surgical site for diagnostic purposes or to view surgical operations occurring through the same scope or a different scope. Surgical operations may include cutting, shaving, debriding, cauterizing, or the like as well as grasping tissues or parts of organs, such as with forceps. Some classes of flexible endoscopes have remotely bendable or steerable tips to enable the surgeon to selectively view and/or operate in a selected direction. With such endoscopes, the surgeon operates a control to cause the tip to selectively curl to reorient the tip. U.S. Pat. Nos. 4,901,142, 6,569,087, and 6,773,395 illustrate exemplary details of such steerable tip flexible endoscopic instruments and are incorporated herein by reference.

Remotely controlled forceps are sometimes used in endoscopic surgery for grasping tissues or tissue structures, for surgical manipulation, incision, biopsy, debridement, or the like and may also be used for purposes such as manipulation of other surgical instruments such as suture needles, sutures, or the like. Thus, the use of forceps in endoscopic surgery requires precise and positive positioning to enable the surgeon to accomplish the needed action.

In a typical remotely controlled forceps instrument, forceps jaws are pivotally mounted in a distal yoke and have scissors links connected to ends proximal ends thereof. The scissors links are connected to an operating cable which extends from the distal yoke through a tubular sheath to a proximately located operating slide or lever engaged with a body or shaft, terminating in a thumb or hand grip. The sheath is fixed to the shaft such that the slide and cable are axially movable relative to the shaft and sheath. The scissors links are arranged so that when the slide is pulled toward the grip, the jaws close. This allows the surgeon to securely grasp the forceps assembly for insertion of the forceps jaws toward the surgical site without the jaws opening and impeding insertion or to retract the assembly from the surgical site, possibly gripping tissue or the like therefrom in the jaws. When the jaws are closed, they are retracted within the yoke which has a diameter not significantly greater than the outer diameter of the sheath. When the slide is pushed relative to the shaft, the jaws are opened. Additional details of remotely controlled endoscopic forceps can be found in U.S. Pat. Nos. 4,763,668 and 5,810,876 which are incorporated herein by reference.

In use of remotely controlled forceps, the surgeon needs to be able to accurately orient the forceps jaws. When remotely controlled forceps are inserted and operated through a rigid endoscopic instrument such as a rigid trephine or trocar, the relationship between the forceps cable and sheath and between the sheath and the lumen of the trephine or trocar is relatively fixed. Therefore, there is usually no binding between the sheath of the forceps instrument and the lumen of the rigid endoscope. However, with flexible endoscopes with remotely bendable tips, there can be frictional interaction of the forceps sheath and the lumen of the scope. The result with conventional forceps is that the surgeon rotates the forceps to orient the jaws at a desired angle, but binding occurs between the sheath and endoscope lumen until a certain resilient force in the forceps sheath overcomes the friction, causing the forceps jaws to rotate suddenly and overshoot the desired angle. Thus, it is often difficult for the surgeon to positively position the jaws of the forceps without frustrating and time consuming trial and error.

### SUMMARY OF THE INVENTION

The present invention provides improvements in endoscopic forceps instruments which enable a surgeon to more accurately and positively orient the jaws of such instruments, particularly with respect to use with flexible endoscopes.

The present invention overcomes the problem of binding of the forceps sheath in a curved flexible endoscope by limiting the torsional resilience of the combination of the forceps sheath and operating cable in a rigidity region of the forceps. The rigidity region of the forceps is defined by a junction between a cable assembly associated with the forceps and a lumen associated with the flexible endoscope and is provided for increasing the deflectional resistance of the forceps. In typical remotely operated endoscopic forceps instruments, the sheath is formed by a steel wire helically wound or wrapped around a spring steel operating cable and gripped at the proximal and distal ends by the proximal body shaft and the distal yoke structure. The arrangement is similar to the configuration of a bicycle hand brake cable arrangement, sometimes referred to as a Bowden cable arrangement. The wrapped steel sheath enables the operating cable to be extended and retracted with low frictional resistance, even when the assembly is curved relatively tightly. The arrangement also provides sufficient axial stiffness to enable the distal end with forceps jaws mounted within a yoke to be inserted through the lumen of a flexible endoscope.

In an embodiment of the improved endoscopic forceps, in order to limit the torsional resilience of the forceps cable assembly of an operating cable and sheath, and thus increase the torsional rigidity thereof, the length of the cable assembly is limited in length. In one embodiment having a cable assembly an outer diameter of approximately 1.5 mm, the length of the cable assembly is no more than about 14 inches (35.5 cm). The action of orienting the forceps jaws is further improved by the material forming the lumen of the flexible endoscope is one which has a low relative frictional coefficient with respect to the steel or stainless steel of which the sheath is formed. Finally, the inner diameter of the endoscope lumen is sized with a relative tolerance to the outer diameter of the forceps tip mechanism and cable assembly to enable free movement through the lumen. By these means, binding of the forceps cable assembly within the endoscope lumen when the endoscope tip is curved is minimized to thereby enable more positive and accurate orientation of the forceps jaws. In one embodiment the elongated cable assembly may be separated from the proximal handle section for distal receipt by the endoscopic instrument and reconnected to the proximal handle section at the port.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is a side elevational view of an embodiment of an endoscopic flexible forceps apparatus with improved torsional rigidity according to the present invention.

FIG. 2 is a greatly enlarged side elevational view of a distal tip of the forceps apparatus and illustrates forceps jaws in a closed relationship.

FIG. 3 is a greatly enlarged side elevational view of the distal tip of the forceps apparatus and illustrates the forceps jaws in an opened relationship.

FIG. 4 is a perspective view of a flexible endoscope instrument with a remotely bendable tip.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

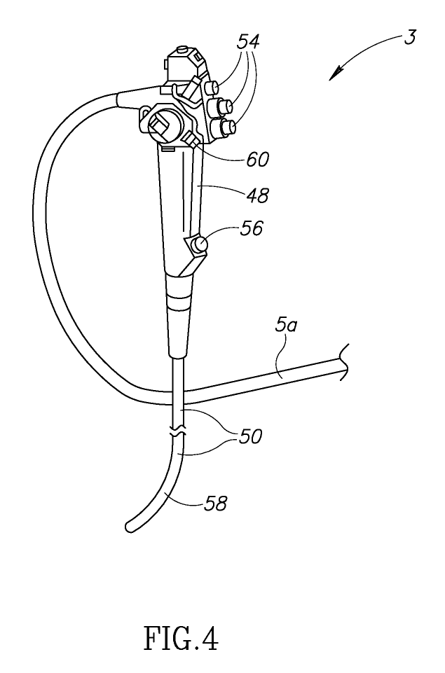

Referring to the drawings in more detail, the reference numeral 1 generally designates an embodiment of a flexible forceps apparatus with improved torsional rigidity according to the present invention. The apparatus 1 is inserted through a flexible endoscope instrument 3 (FIG. 4) for grasping structures within an endoscopic, arthroscopic, laparoscopic, or similar type of surgical site. The forceps apparatus 1 generally includes a proximal handle section 8, an elongated cable assembly 10, and a distal tip section 12 including a pair of opposed forceps jaw members 14.

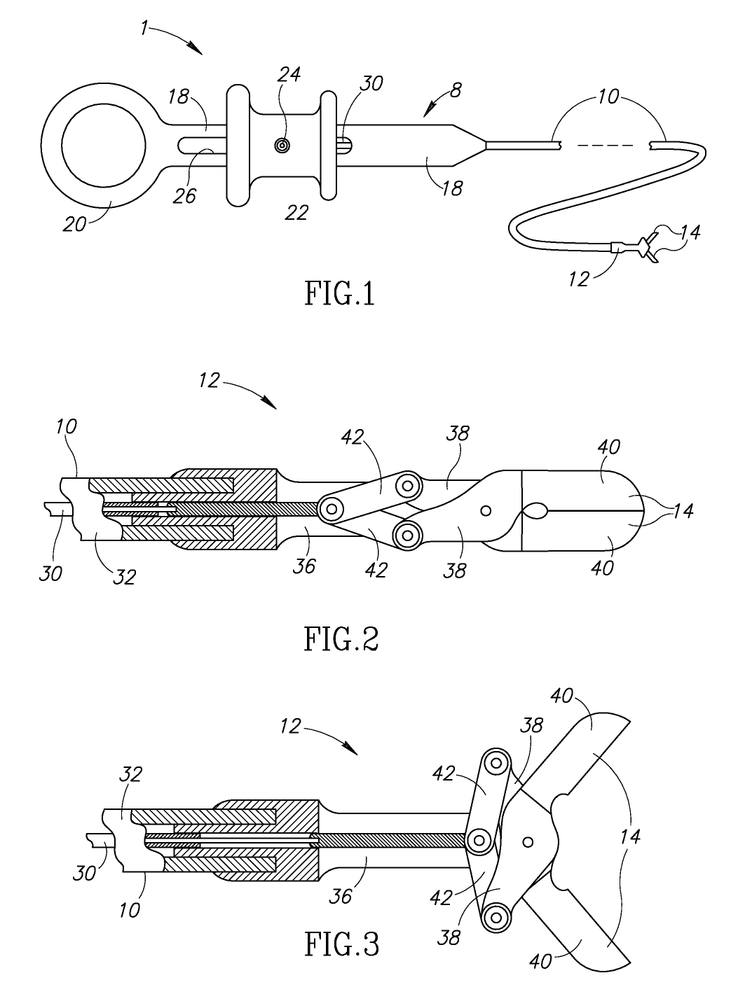

Referring to FIG. 1, the illustrated handle section 8 includes an elongated rod shaped handle frame 18 terminating proximately in a thumb ring 20. An operating spool 22 is slidably mounted on the frame 18 and has an attachment post assembly 24 including a set screw extending through an elongated slot 26 formed through a section of the frame 18. A proximal end of a jaws operating cable 30 is attached to the post 24 by means of the set screw. A proximal end of a tubular cable sheath 32 of the cable assembly 10 is secured to a distal end of the handle frame 18. Although not illustrated, the sheath 32 can be formed by a wire of round or flattened cross section which helically wound or wrapped about the cable 30.

In one embodiment, the spool 22 may include a concave parabolic surface for operative gripping. In another embodiment, the cable assembly 10 is removably attached to the proximal handle section 8 for distal receipt by the endoscopic instrument 3 and reconnection to the proximal handle section 8 at an instrument port 56 associated with the endoscopic instrument 3.

Referring to FIGS. 2 and 3, the tip section 12 includes a pair of forceps jaws mounting yoke 36 which is secured to a distal end of the cable sheath 32. The yoke 36 has the forceps jaw members 14 pivotally connected between legs thereof. Each jaw member 14 is formed by a proximal jaw lever 38 and a distal jaw 40. Although not illustrated, the jaws 40 can be provided with serrated or toothed gripping surfaces. Each jaw lever 38 is pivotally connected to a respective scissors link 42, and ends of the links 42 are pivotally connected together and secured to a distal end of the operating cable 30. Retraction of the cable 30 into the sheath 32 pulls the scissors links 42 in a proximal direction and thereby pivots the jaws 40 toward closed positions. Conversely, extension of the cable 30 in a direction out of the sheath 32 pushes the scissors links 42 in a distal direction and pivots the jaws 40 toward open positions.

In operation of the forceps apparatus 1, the surgeon holds the handle section 8 with a thumb extended through the thumb ring 20 and with the operating spool 22 held between the forefinger and middle finger or, alternatively, between the middle finger and ring finger. Movement of the spool 22 toward the thumb ring 20 pulls the cable 30 in a retraction direction, thereby closing the forceps jaws 40. Conversely, movement of the spool 22 away from the thumb ring 20 extends the cable 30, thereby opening the forceps jaws 40.

Referring to FIG. 4, the illustrated flexible endoscope apparatus 3 includes a handle section 48 to which is attached an elongated composite scope assembly 50 which is inserted through an incision or through a patient's body orifice by a surgeon. The scope assembly 50 typically includes (not shown) one or two fiberoptic bundles carrying light from a remote light source, a coherent fiberoptic bundle carrying an image viewed within the surgical site, one or more fluid carrying lumens, and an instrument lumen for the insertion of endoscopic surgical instruments, such as the forceps apparatus 1. The image carried by the coherent fiberoptic bundle illuminates an image array within the handle section 48, and a video signal is communicated to a remote video monitor by a video cable 52 for direct viewing by the surgeon and possible recording. The handle section 48 is provided with one or more fluid controls 54, for controlling the injection of fluids into the surgical site or suction, and the instrument or forceps port 56, through which a surgical instrument such as the forceps apparatus 1 can be inserted. The illustrated scope assembly 50 has a bendable or steerable tip 58 which is selectively controlled by the surgeon by rotation of a steering control 60 mounted on the handle section 48.

When use of the forceps apparatus 1 with the endoscope instrument 3 is needed, the surgeon grasps the handle section 8 and pulls the spool 22 toward the thumb ring 20 to close the jaws 40 and to increase the axial rigidity of the cable assembly 10. The tip section 12 is inserted into the forceps port 56 of the instrument 3, followed by the cable assembly 10. Insertion is continued until the tip section 12 reaches the surgical site. The tip section 12 and cable assembly 10 must pass through any curves in the composite scope 50. Once the tip section 12 is at the surgical site, the surgeon must carefully position and orient the jaws 40 for use in grasping whatever structure requires manipulation. Correction of the axial position of the tip section 12 is a simple matter of extending or retracting the apparatus 1 with respect to the endoscope instrument 3. However, angular correction of the orientation of the jaws 40 is sometimes resisted by frictional contact of the cable sheath 32 with inner walls of the lumen (not shown) through which the cable assembly 10 extends, particularly at locations of bends in the composite scope 50. Torsional resistance to rotation of the cable assembly 10 is resisted until overcome, at which point, the tip assembly 12 suddenly jumps. Thus, angular correction of the jaws 40 sometimes overshoots the desired orientation.

In order to overcome orientation problems with the forceps jaws 40, improvements are made in the cable assembly 10 to increase its rotational rigidity without affecting the overall flexibility of the cable assembly 10. The length of the cable assembly 10 from a distal end of the handle frame 18 to the tip assembly 12 is limited to control the torsional resilience of the cable assembly 10. In the illustrated apparatus 1, the length is limited to about 14 inches (35.5 cm). The illustrated cable sheath 32 is formed by a stainless steel wire wrapped helically about the operating cable 30. The desirable length of the cable assembly 10 may be different for other materials and types of construction of the cable sheath 32. Additionally, the outer diameter of the cable sheath 32 must have a desired clearance within the instrument/forceps lumen within the composite scope 50. Finally, the relative coefficient of friction between the material forming the cable sheath 32 and the forceps lumen must be low to further reduce frictional binding between the cable sheath 32 and the forceps lumen.

To improve the torsional rigidity region 16 associated with the apparatus 1, it is desirable to decrease the accumulation of stress leading to the torsional deformation of the cable assembly 10 and decrease the frictional resistance generated between the cable assembly 10 and the flexible endoscope 3, improving the transmission of the flexible forceps 1 to the surgical site. Stated differently, the torsional rigidity region 16 can be improved by increasing the torsional rigidity and decreasing the flexural rigidity. In one embodiment the improved torsional rigidity region 16, defined by a junction between the cable assembly 10 including the sheath 32 and cable 30 and a lumen 50a associated with the elongated composite scope assembly 50, may be provided, the sheath 32 having an outer sidewall with a reduced frictional surface for being received by the lumen 50a wherein the lumen 50a and the sheath 32 have complementary properties for increasing the deflectional resistance of the distal tip section 12 while preventing the cable assembly 10 from binding within the composite scope assembly 50.

Generally, the distal end of the composite scope assembly 50 is separated a distance from the distal tip section 12, which itself is further separated from the surgical site. Depending upon the characteristics and configuration of the received flexible forceps 1, the separation distance is between X and Y millimeters, where a distance less than X or greater than Y would provide non-optimal insertional rigidity for the flexible forceps 1. The change between X and Y is represented by Δx and the moment of inertia corresponding to the composite scope assembly outer surface 50 corresponds to mr² where m is the mass of the composite scope assembly 50 and r is the corresponding radius. The moment of inertia related to the flexible forceps 1 therefore corresponds to (mL²)/3 where L is the length, Δx, and m is the mass of the flexible forceps 1. In this case, the flexible forceps 1 has an exponentially greater moment of inertia based upon Δx, however, as Δx increases the moment decreases resulting in a corresponding loss of rigidity of the flexible forceps 1. Stated another way, as the distal tip section 12 extends farther out of the distal end of the composite scope assembly 50, the flexible forceps 1 become less torsionally rigid. In addition, by decreasing Δx, cartilage or other tissue associated with the surgical site may be damaged by a corresponding increased rigidity associated with the flexible forceps 1 extending past the composite scope assembly distal end 50.

It is to be understood that while certain forms of the present invention have been illustrated and described herein, it is not to be limited to the specific forms or arrangement of parts described and shown.

What is claimed and desired to be secured by Letters Patent:

1. An improved medical transmission apparatus for transmitting a flexible forceps through one of a plurality of lumens associated with an elongated composite scope assembly towards a surgical site wherein the improved medical transmission apparatus comprises:
an increased torsional rigidity region for increased deflectional resistance of said forceps and being defined by a junction between a sheath associated with said forceps and a lumen extending cylindrically along said elongated composite scope assembly;
a portal entry associated with said elongated composite scope assembly having a diameter sized for receiving and transmitting a distal tip section associated with said forceps therethrough; and
a cylindrical clearance presented at said increased torsional rigidity region between an outer sidewall associated with said sheath and an inner surface associated with said lumen, said cylindrical clearance directing said forceps towards said surgical site.

2. The improved medical transmission apparatus of claim 1 that further comprises:
a cable associated with said forceps and extending through said sheath for communication with said distal tip section.

3. The improved medical transmission apparatus of claim 2 wherein said cable is no longer than 14 inches.

4. The improved medical transmission apparatus of claim 2 that further comprises:
a proximal handle section including a handle frame and an operating spool having a concave parabolic surface, said spool being associated with said handle frame for maneuvering said forceps;
said handle frame extending forwardly from a thumb ring, through said operating spool and communicating with said distal tip section by said sheath; and
an attachment post associated with said spool and extending through an elongated slot associated with said handle frame for sliding said spool along said handle frame to control said distal tip section.

5. The improved medical transmission apparatus of claim 4 that further comprises:
a forceps jaws mounting yoke associated with said distal tip section extending distally from said sheath and having a pair of proximal jaw levers pivotally mounted on said yoke for pivotally moving a pair of jaw members between an open jaw position to a closed jaw position;
a pair of scissor links extending distally from said cable and being pivotally connected to said jaw levers for pivotal movement between a triangular pivot position associated with said closed jaw position to a linear pivot position associated with said open jaw position; and
said operating spool being operatively connected to said cable for slidable movement of said scissor links.

6. The improved medical transmission apparatus of claim 5 that further comprises:
a cable assembly including said cable and said sheath and being attached to said distal tip section by said cable and by said sheath for communication between said proximal handle section and said distal tip section; and
said cable assembly being removably connected to said proximal handle section for inserting said cable assembly and distal tip section through an opening associated with said composite scope, the lumen therein and said port and onto said proximal handle section.

7. The improved medical transmission apparatus of claim 6 that further comprises said cable being removably attached to said attachment post and secured thereon by a set screw.

8. The improved medical transmission apparatus of claim 2 wherein said cable is composed of a material for transmitting an electrical current to said jaw members for electro-cauterization within said surgical site.
